(19) United States
(12) Patent Application Publication
(10) Pub. No.: US 2018/0344683 A1
(43) Pub. Date: Dec. 6, 2018

(54) DRUG FOR PREVENTING AND TREATING ALOPECIA

(71) Applicant: Xinfang DUAN, Nanyang (CN)

(72) Inventor: Xinfang DUAN, Nanyang (CN)

(21) Appl. No.: 15/861,621

(22) Filed: Jan. 3, 2018

(30) Foreign Application Priority Data
May 31, 2017 (CN) 201710397535.0

(51) Int. Cl. A61K 31/352; A61K 33/42; A61K 31/341; A61K 31/191; A61K 31/19; A61K 31/194; A61K 33/10; A61K 31/7048; A61P 17/14

(52) U.S. Cl. CPC A61K 31/352; A61K 33/42; A61K 31/341; A61K 31/191; A61P 17/14; A61K 31/194; A61K 33/10; A61K 31/7048; A61K 31/19

(57) ABSTRACT

The present disclosure provides a drug for preventing and treating alopecia and belongs to the technical field of pharmaceutical products. The drug for preventing and treating alopecia comprises the following active ingredients: a composition including more than one of flavonoid compound with effects of decreasing capillary permeability, calcium and vitamin C. The composition in the present disclosure can be adopted to achieve aims of effectively preventing and treating alopecia and promoting hair growth, and is safe in property, wide in universality, obvious in curative effects and suitable for treatment of androgenetic alopecia.

### TECHNICAL FIELD

The present disclosure belongs to the technical field of pharmaceutical products, and particularly relates to a drug for preventing and treating alopecia.

### BACKGROUND

Alopecia is a skin disease featured by a hair decrease. The alopecia includes many types such as alopecia areata, alopecia cicatrisata, papular alopecia, alopecia capitis totalis, alopecia caused by mental factors, alopecia caused by nutrient deficiency, alopecia seborrhoeica, alopecia caused by medical chemotherapy, androgenetic alopecia and the like. It can be seen from many types of alopecia that many reasons cause the alopecia, but most of the reasons of the alopecia are unclear, and no drug can be used for treatment. As is well-known, the androgenetic alopecia is one type of the alopecia with the widest involved crowd, the highest research input and the longest research time around the world at present. Since the androgenetic alopecia often occurs in youth and greatly harms nature of young people loving beauty, and endless trouble and pain may be inevitably brought to them. Just because of this, mankind has always been devoted to conquering such an alopecia disease for a long time. Thousands of years ago, ancient Egyptians applied hippo dung to scalp for trying to treat baldness; and 400) years ago, Hippocrates, the pre-Hellenic Father of Medicine, applied pigeon dung to the scalp for hoping to cure the alopecia. Unremitting exploration is also made in the present field in Chinese medicine. As recorded in Compendium of Materia Medica, sophora branches are burned to ash, and the ash is applied to the scalp with lard oil for curing the baldness. However, practice has proved that the alopecia is not be effectively controlled by the above methods. Moreover, the andmgenetic alopecia has a trend of scope expansion at present.

At present, the most effective method recognized in the field for treating the androgenetic alopecia is external application of minuodier lotion and oral administration of finasteride (Chen Shuxin, Li Jiehua, Mo Yufang. Observation of Clinical Curative Effect for Treatment of Androgenetic Alopecia in Combination of Minuodier Lotion and Finasteride [J]. International Medicine & Health Guidance News, 2006, 12(6):64-65.) According to an experimental study, minuodier is added into in-vitro culture of hair follicles, so that growth time of the hair follicles may be increased. The minuodier stimulates and maintains the growth of the hair follicles and prolongs a growth period of the hair follicles, so that functions of developing and increasing micro hair follicles and promoting hair papilla vascularization are realized, so that the minuodier plays an important role in treatment of the alopecia. Finasteride is a 4-azasteroids compound and is a specific inhibitor of intracellular enzyme-II type 5α-reductase in a process of metabolizing testosterone into stronger 5α-dihydrotestosterone. However, so far, it has been proved that the minuodier and the finasteride have obvious defects. Topical application of the minuodier may cause side effects of a certain degree. The side effects include rash, local inflammation, headache, hirsutism and the like, while oral application of the finasteride is determined to cause hormonal dysfunction with a potential negative effect on sexual life, and the finasteride has genetic and reproductive toxicity.

At present, there is an urgent need of researching and developing a product which can effectively prevent the alopecia and promote hair growth without causing any obvious negative effects in use.

A patent with a publication number of CN1138460 provides a novel application of a calcium-enriched calcium product taking calcium as a raw material; an application of taking the calcium product as a health care product or a drug with effects of securing essence and tonifying kidney, a novel application of taking the calcium product as an agent for preventing and treating alopecia and increasing hair, and a novel application of taking the calcium product as a drug for treating acquired diabetes mellitus, thereby achieving effects of improving a human metabolism function, promoting blood circulation, building the body, increasing and protecting hair and preventing and treating diseases. The disclosure may be an active calcium product made from a calcium raw material of 300-3000 meshes or a calcium product of pearl powder. Through calculation of pure calcium, a dose is selected to be 200-3000 mg per day. An emphasis of the disclosure is a supplement of calcium. A series of basic diseases including hair loss caused by calcium deficiency or kidney deficiency are prevented or treated by supplementing the calcium, just as recorded in the disclosure as follows: “novel application of a high-calcium calcium product taking calcium as a raw material”, “high-dose calcium supplement is discovered to have obvious curative effects for securing essence, tonifying kidney, contributing to normally transport nutrition in blood and preventing and treating hair loss”. However, based on etiology and pathogenesis of the androgenetic alopecia, long-term medication is needed for radical cure of the alopecia, while side effects caused by single long-term administration of large dose calcium should never be ignored. Peng Jinxiang et al. (Peng Jinxiang, Wang Ting. Research Progress of Relationship between Calcium Supplement and Cardiovascular Diseases [J]. Chinese Journal of Geriatric Care, 2014(4):65-66) find through research that long-term large dose calcium supplement may increase risk of suffering from the cardiovascular diseases.

A patent with a publication number of CN101102763 provides a composition which has effects of stimulating growth of dermal papilla cells and promoting growth of the hair follicles and contains vitamin C derivatives. The vitamin C derivatives contained in the composition serve as active ingredients and are selected from L-ascorbyl-2-phosphate magnesium salt, L-ascorbyl palmitate and L-ascorbyl stearate. The composition can treat the alopecia, and the agent can stimulate the growth of the dermal papilla cells and/or the hair follicles. Meanwhile, the disclosure further points out that the vitamin C is rapidly oxidized and degraded in an aqueous solution, particularly under a general culture condition, so the application of the vitamin C is limited. Comparatively speaking, the vitamin C derivatives have better stability. However, the disclosure does not relate to an applicable solution for treating the androgenetic alopecia, and any instance report that the androgenetic alopecia is successfully treated by taking the vitamin C derivatives as the active ingredients is not seen

Flavonoid compounds refer to a type of polyphenol natural products with wide distributions in plants and have a structure of 2-phenylchromone. The flavonoid compounds are generally bound with sugar into glycosides in plant bodies, and a small part of the flavonoid compounds exist in a form of a free state (aglycone). The flavonoid compounds are contained in most of the plant bodies, and play an important role in the aspects of growth, development, flowering, bearing fruits, bacteria resistance and disease prevention of plants. The flavonoid compounds have a history of more than 300 years from discovery up to now, and novel flavonoid compounds are separated from natural plants per year. The flavonoid compounds have structural diversity. Most of the flavonoid compounds have three rings A, B and C, and according to substitution positions of the ring B as well as differences of ring formation, oxidation and substitution manners of the ring C, the flavonoid compounds may be divided into flavonoids, flavonols, chalcones, isoflavones, dihydroflavones, dihydroflavonols, dihydrochalcones, isoflavanones, biflavones, aurones, flavanoids, isoflavanes, anthocyans, xanthones and the like. Since the flavonoid compounds have many varieties, different structures and diversified biological activities, research on the biological activities of the flavonoid compounds is always a human research hot topic. At present, a great deal of researches show that the flavonoid compounds have physiological activities such as antibacterial and antivirus properties, oxidation resistance, antitumor property, anti-inflammatory property, decrease of blood sugar, osteoporosis resistance, rheumatism resistance, action on a cardiovascular system and the like. Moreover, the current research shows that the isoflavone, flavanone, chalcone and other flavonoid compounds have treatment effects on different types of the alopecia. However, long-term excessive consumption of certain flavonoid compounds is proved to produce obvious side effects (Lu Yan, Li Junsheng, Yan Liujuan. Potential Hazard from Excessive Consumption of Flavones [J]. Lishizhen Med Mater Med Res, 2009, 20(12):3079-3081).

A patent with a publication number of CN102641353A provides a pilatory enriched in plant flavonoid compound. The pilatory is prepared from the following raw materials in parts by weight: 30-90 parts of plant extract and 0.3-0.9 part of synergist, wherein the plant extract is calculated based on total flavones, and a weight content of the total flavones is 10-50%; and the synergist is prepared by mixing nicotinamide, vitamin B6 and zinc sulfate according to a weight ratio of (1-3):(1-3):(1-3). The product in the present disclosure achieves an effect of inhibiting production of reductase causing the alopecia by utilizing the flavonoid compound in the plant extract. According to a synergistic effect between the synergist and the plant extract in a special ratio, active ingredients in the plant extract may well permeate into scalp cuticle and sebum, thereby achieving stronger effects of growing, fixing and blackening hair, further enhancing effects of preventing the alopecia and promoting hair growth and fundamentally preventing the alopecia. In the disclosure, the nicotinamide, the vitamin B6 and the zinc sulfate are combined to serve as the synergist; the synergist and the plant extract are combined to be medicated; the plant extract contains 10-50% of the total flavones; and medicinal raw materials have complicated composition. A principle of growing, fixing and blackening the hair in the present disclosure is to inhibit the production of the reductase causing the alopecia, and the above effects are enhanced by virtue of the synergist. The plant extract in the present disclosure is extracted from multiple traditional Chinese medicinal materials. The components are unclear, and an achieved pharmacological effect is also unclear.

### SUMMARY

In view of descriptions mentioned above, a technical problem to be solved in the present disclosure is to provide a natural composition for effectively preventing and treating alopecia and promoting hair growth. The drug is safe in property, wide in universality and obvious in curative effects.

In order to achieve the aims, a technical solution adopted in the present disclosure is as follows.

A drug for preventing and treating alopecia comprises the following active ingredients: a composition including more than one of flavonoid compound with effects of decreasing capillary permeability, calcium and vitamin C.

Preferably, the flavonoid compound refers to quercetin, kaempferol or kaempferide.

Preferably, the flavonoid compound refers to dihydroquercetin.

Preferably, the flavonoid compound refers to rutin, apigenin, apiin, diosmetin or diosmin.

Preferably, the calcium is milk calcium, eggshell calcium, oyster calcium, seaweed calcium, coral calcium, pearl calcium, bone calcium, calcium lactate, calcium acetate, calcium gluconate, calcium citrate, calcium phosphate, calcium carbonate, calcium pantothenate or calcium chloride.

Preferably, the active ingredients of the drug for preventing and treating alopecia comprise the flavonoid compound and the calcium, wherein a mass ratio of the flavonoid compound to the total active ingredients is less than ⅓.

Preferably, the active ingredients of the drug for preventing and treating alopecia comprise the flavonoid compound and the vitamin C, wherein a mass ratio of the flavonoid compound to the total active ingredients is less than ½.

Preferably, the active ingredients of the drug for preventing and treating alopecia comprise the flavonoid compound, the calcium and the vitamin C, wherein a mass ratio of the flavonoid compound to the total active ingredients is less than ⅓.

Preferably, the active ingredients of the drug for preventing and treating alopecia comprise the flavonoid compound, the calcium and the vitamin C, wherein a weight ratio of the flavonoid compound to the calcium to the vitamin C is 1:(1-10):(1-10).

Preferably, the active ingredient of the drug for preventing and treating alopecia comprise the quercetin, the milk calcium and the vitamin C, wherein a weight ratio of the quercetin to the milk calcium and to the vitamin C is 1:(1-10):(1-10).

Compared with the prior art, the present disclosure has beneficial effects as follows.

The drug for preventing and treating the alopecia in the present disclosure comprises the following active ingredients: the composition including more than one of the flavonoid compound with effects of decreasing capillary permeability, the calcium and the vitamin C. The “alopecia” in the present disclosure refers to androgenetic alopecia, and the phenomenon of the androgenetic alopecia are as follows. Hair grows well before hair loss, and is the same in thickness, density and color as those of normal people; the hair loss often starts from about 20 years old, and symptoms are obvious on foreheads and tops; during a serious period of hair loss, about 100-200 pieces of hair are lost; even so, a small amount of hair still grows, while newly grown hair is relatively thin, soft and yellow. In decades of study, the applicant considers that regards to the androgenetic alopecia, a physiological change is caused in a hair loss region under an action of natural selection, and capillary permeability is increased. So that more ceaseless energy is provided for accelerated division and proliferation of hair follicles and a metabolic cycle of the hair is further shortened, thereby enabling the hair loss in advance. About 100-200 pieces of hair are lost per day during the serious period of hair loss for a patient suffering from the androgenetic alopecia. According to the statistics, a quantity of hair of a person is about 30000, and the hair will be totally lost in less than a year according to the above-mentioned hair loss speed. However, it is not the case. Generally the hair of the patient suffering from the androgenetic alopecia is completely lost or become sparse within ten years or even decades, that is, hair follicle cells of the patient suffering from the androgenetic alopecia always do not deviate physiologically, but a speed of division and proliferation of the hair follicle cells is increased. Therefore, the applicant considers that if the capillary permeability of the head can be decreased and energy supply of the hair follicle cells can be decreased to transfer the hair follicle cells into a normal or nearly normal division and proliferation state, then a hair loss state of the patient suffering from the androgenetic alopecia may be controlled. But meanwhile, it must be recognized that the action of natural selection is a continuous, long-term and ongoing process. The hair serving as one part of a human body inevitably follows physiological metabolism. Although the diversion of the hair follicle cells on the head is transferred to be normal or nearly normal, once drug administration is stopped, a fact that the hair is still lost again cannot be ignored. Therefore, the patient suffering from the androgenetic alopecia must take the drug for a long time and even throughout his life. Based on this, safety of the drug is of great importance. Moreover, the applicant finds through research, that a dose of the drug and a growing and retaining amount of the hair present an obvious dose-effect relationship. The greater the dose, the higher the retaining and growing amount of the hair is However, side effects brought by large-dose administration of the drug must be brought to attention. In order to adapt to the fact that the drug should be taken for a long time and even throughout the life, dose control of the drug is also of great importance.

The drug for preventing and treating the alopecia is researched in the present disclosure under guidance of the above theory. Firstly, the flavonoid compound with the effects of decreasing the capillary permeability are natural extracts used in the present disclosure; the calcium and the vitamin C are common nutritional ingredients of health care products; the components are safe; and the flavonoid compound with the effects of decreasing the capillary permeability, the calcium and the vitamin C often exist in fruits, vegetables and other foods for daily consumption in a coexistence relationship. Secondly, the applicant finds through research that the flavonoid compound, the vitamin C or the calcium taken alone may achieve the effects of preventing and treating the androgenetic alopecia and promoting hair growth. However, even though the flavonoid compound, the vitamin C or the calcium can be adopted separately for medical treatment, it is limited to or only suitable for patients suffering from mild or early androgenetic alopecia, and the drug should be intermittently used. For relatively severe or severe patients suffering from androgenetic alopecia, growth and recovery capability of the hair is generally low. Single medication of the above drugs must be persisted for a long time and even a larger dose is used, but safety is comparatively lower. For example, as everyone knows, large-dose administration of the vitamin C easily causes a side effect of gingival bleeding. A long-term large-dose administration of the calcium may increase a risk of cardiovascular diseases (Peng Jinxiang, Wang Ting. Research Progress of Relationship between Calcium Supplement and Cardiovascular Diseases [J]. Chinese Journal of Geriatric Care, 2014(4):65-66). In view of the above reasons, from the viewpoint of safety, long-term large-dose administration of single component is inadvisable. However, in the research process, the applicant accidentally discovers that more than two of the flavonoid compound, the calcium and the vitamin C are combined for use, are safe to be taken for a long time while controlling or decreasing a dose of each component per day and do not have any obvious side effect, and obvious effects of preventing and treating the androgenetic alopecia and promoting the hair growth are achieved. Particularly, when the three components including the flavonoid compound, the calcium and the vitamin C are used simultaneously, the treatment effect is the most obvious. No side effect is caused when the drug is continuously taken for a long time under the condition that the dose of each drug is controlled. An unexpected synergistic effect is achieved, thereby avoiding possible side effects brought by single medication. The flavonoid compound, the calcium and the vitamin C adopted in the present disclosure have the effect of decreasing the capillary permeability, which is closely related to a mechanism for preventing and treating the androgenetic alopecia in the present disclosure. Targeting effects of the three substances are very clear, and targets point directly at capillary vessels. When the effects of the substances are achieved, the capillary permeability is decreased, and energy supply is also decreased. An accelerated division and proliferation state of the hair follicle cells in a calvarium hair loss region is immediately transferred into a normal or nearly normal division and proliferation state due to an energy decrease, and then hair loss is controlled. More importantly, one part of the lost hair can still restore vitality. Meanwhile, it is indicated that although the lost hair cannot be seen from an appearance, the hair follicle cells may still execute an instruction of accelerated division and proliferation from the natural selection, while division activities of the hair follicles return to a normal state when the energy supply is decreased, metabolism restores to or is close to a normal level, and the hair naturally restores vitality again. Moreover, facts prove that the three substances, that is, the flavonoid compound, the calcium and the vitamin C, are matched with one another and achieve synergistic effects, thereby increasing treatment effectiveness, obviously increasing the safety on a basis of increasing the dose compared with a dose of a single substance, avoiding possible side effects brought by single administration of one component and indicating an increase of the hair retaining amount along with an increase of the dose. When the three substances are matched to be used, a sharp lost of hair may be reversed of a patient suffering from the androgenetic alopecia. A sharp hair loss may be controlled when the drug is taken for half a month, regardless of an age in the initial hair loss, or if the hair is lost to be sparse before complete loss. The hair may obviously grow when the drug is taken for 2-4 months. The hair may cover the scalp when the drug is taken for 1-2 years. The hair loss is controlled due to persistent drug administration, and hair growth returns to normal. In the present disclosure, it can be seen from the composition of a formula that it has all characteristics of new drug development standards, that is, “safety, effectiveness, controllability, universality and low price”. It should be pointed out that, regardless of ages of patients with the hair completely lost, the hair is difficult to restore to cover the scalp before the drug administration. However, if the drug in the present disclosure can be persistently taken, unexpected effects can also be achieved. The composition in the present disclosure is a hair-loss-preventing hair-growing drug which can be taken for a long time, has a health effect and does not have any toxic or side effect. A marked effective rate for preventing and treating the androgenetic alopecia in the present disclosure reaches 100%.

### BRIEF DESCRIPTION OF THE DRAWINGS

The present disclosure is described below in detail in combination with drawings.

FIG. 1 is a simulated diagram of a formation mechanism of androgenetic alopecia.

FIG. 2 are effect pictures (A) and (B) of a certain patient suffering from androgenetic alopecia before and after receiving treatment of a composition in the present disclosure.

### DETAILED DESCRIPTION OF DISCLOSED EMBODIMENTS

In order to better understand the present disclosure, contents of the present disclosure are further clearly illustrated below in combination with embodiments, but protection contents of the present disclosure are not limited to embodiments below. In description below, a great quantity of specific details are given to provide a thorough understanding for the present disclosure. However, it is apparent for those skilled in the art that the present disclosure may be implemented without one or more of these details.

Alopecia described in the present disclosure refers to androgenetic alopecia, also called male alopecia or male baldness. Particularly in men, the disease is often called male pattern alopecia. Hair is lost in a clear boundary form from positions above both temples. As time goes by, a hairline recedes to form a characteristic “M” or “U” shape. Hair on the head crest is thinned. Generally hairlines surrounding side surfaces and a back surface of the head are retained only. A hair loss form of women is different from that of the male pattern alopecia. In women, all hair in the scalp becomes thinner and thinner, but the hairlines do not recede. The androgenetic alopecia in women hardly causes alopecia totalis on the top of the head.

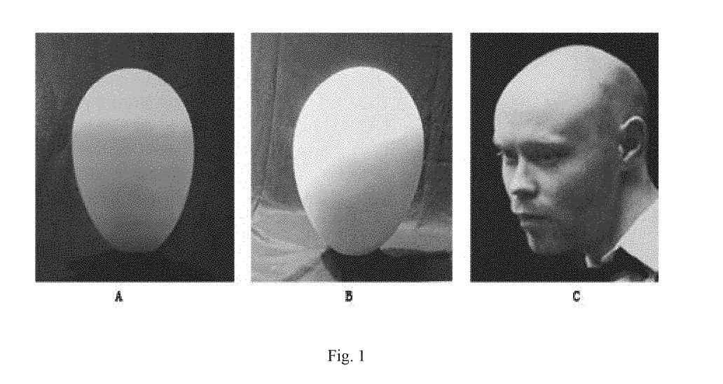

It should be noted that, such a name of the “androgenetic alopecia” is a habitual name formed over the years, but so far, the androgenetic alopecia is not clearly defined medically. Moreover, most of researches do not support effects of a single factor, that is, androgen, on alopecia. An indisputable fact is that, the androgenetic alopecia cannot explain why the hair is lost only on the top of the head, but not on tempora on two sides, above and behind ears and below cerebric occiput. It cannot be denied that, an evolution viewpoint may explain such a peculiar phenomenon of human beings. As is well-known, human skin converts vitamin D. The vitamin D is also called sunshine vitamin and is converted by irradiating skin using sunshine. An effect of the vitamin D in the body is to help calcium absorption, while calcium is an essential substance for maintaining bone health. Since human beings at an ice age wear warm clothes sewed by animal skin and strongly settle down in the high northern latitudes on earth, skin is tightly covered by the clothes, and then the vitamin D for maintaining human bone health meets an unprecedented challenge. To this end, natural selection determinedly enables hair to be lost for protecting the top of the head. In fact, skin on the top of the head is fully exposed in sunshine for converting the vitamin D needed for maintaining the bone health. In order to enable the above problem to be intuitive, a model shown in FIG. 1 is used for illustrating in the present disclosure. The head of a person looks like an inverted egg-like elliptic structure and is large in top and small in bottom. In FIG. 1, A shows an effect picture for simulating straight sunshine on the head. It can be seen from the picture that, when light directly irradiates the model shown as A in FIG. 1 from top to bottom, the light can only irradiate in a maximum perimeter position of the egg-like elliptic structure, and a part below the maximum perimeter position is in the shadow, thereby forming two parts with a clear light-shade boundary on an upper part (the top) and a lower part. Coincidentally, a patient suffering from the androgenetic alopecia (FIG. 1C) just also is as follows: a peculiar baldness phenomenon is formed by taking a maximum perimeter of the head as a boundary, while hair below the maximum perimeter is not lost till old. An actual irradiation effect (FIG. 1B) obtained by placing the model shown as F in FIG. 1 in the sunshine is completely consistent with a baldness effect (FIG. 1C).

Therefore, an androgenetic alopecia phenomenon is a product of evolution and is genuine physiological alopecia rather than pathological alopecia. The above phenomenon fully proves a viewpoint of the present disclosure that: hair loss of the patient suffering from the androgenetic alopecia is realized by accelerating division and proliferation activities of hair follicle cells and by shortening a metabolic cycle in the natural selection. The hair may be completely lost in advance and does not deviate physiologically from division and proliferation of the cells. Certainly, accelerated division and proliferation of the cells still need support of energy. Therefore, capillary permeability is decreased by a drug; and a transport amount of energy needed by the accelerated division and proliferation to the hair follicle cells is decreased. The hair follicle cells including stem cells may naturally return to a normal or nearly normal division and proliferation orbit. The hair is not sharply lost any more, and a great majority of the lost hair can still restore vitality. Just because of this, in-vitro experimental simulation and exploration of the androgenetic alopecia make no sense. Apparently, copying of a physiological environment consistent with evolutionary morphology is unrealistic. A term “prevention of alopecia” in the present disclosure includes prevention or inhibition of hair loss related to alopecia before the alopecia occurs. A term “treatment of alopecia” in the present disclosure includes decrease of severity of the hair loss related to the alopecia or decrease of a degree of the hair loss related to the alopecia.

A drug for preventing and treating the alopecia in the present disclosure comprises the following active ingredients: a composition including more than one of a flavonoid compound with effects of decreasing capillary permeability, calcium and vitamin C.

The flavonoid compound used in the present disclosure is the flavonoid compound with the effects of decreasing the capillary permeability. As a preferred embodiment of the present disclosure, the flavonoid compound is selected from quercetin, kaempferol or kaempferide, wherein the quercetin is also called meletin, 2-(3,4-dihydroxyphenyl)-3,5,7-trihydroxy-4H-1-benzopyran-4-one, 3,3′,4′,5,7-pentahydroxyflavone. A structural formula of the quercetin is

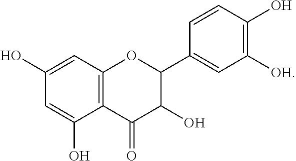 [SMILES: O=C1c2c(O)cc(O)cc2OC(c2ccc(O)c(O)c2)C1O]

The quercetin widely exists in angiosperms, such as threevein aster, euphorbia lunulata, berchemia lineata, folium rhododendri daurici, seguin loquat, rhododendron mariae, rhododendron micranthum, Japanese ardisia herb and apocynum venetum. The quercetin is an aglucone. The aglucone is often bound with sugar to exist in a form of glycoside, such as quercitrin, rutin, hyperoside and the like. The quercetin is prepared by grinding bark of quercus plants of fagaceae into powder, washing with hot salt water, extracting with dilute ammonia water, neutralizing with dilute sulfuric acid, boiling the filtrate, and separating a crystal. The quercetin may be extracted from liliaceous plants such as alliumcepa and the like with 95% of ethanol, and may also be prepared by hydrolyzing rutoside (rutin) extract and quercitrin, isoquercitrin, foeniculin, hyperoside, quercimeritrin, meratin and the like by virtue of rutoside lytic enzymes or an acid aqueous solution.

The kaempferol is also called kaempferol-3, kaempferide, nimbecetin, kaempferol, nimbecetin III, and has a molecular structure of C₁₅H₁₀O₆ and a structural formula of

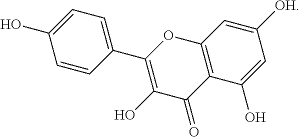 [SMILES: O=c1c(O)c(-c2ccc(O)cc2)oc2cc(O)cc(O)c12]

The kaempferol mainly comes from rhizomes of zingiberaceae rhizoma kaempferiae and widely exists in various fruits, vegetables and drinks. At present, pure kaempferol has been extracted from tea leaves, broccoli, witch hazel, propolis and grapefruits.

The kaempferide is also called 3,5,7-trihydroxy-4′-methoxyflavone; kaempferol-4′-O-methyl ether, has a structural formula of

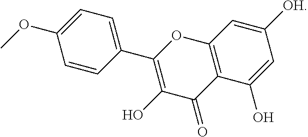 [SMILES: COc1ccc(-c2oc3cc(O)cc(O)c3c(=O)c2O)cc1]

and is extracted from the rhizomes of the zingiberaceae rhizoma kaempferiae.

As another preferred embodiment of the present disclosure, the flavonoid compound is selected from dihydroquercetin. The dihydroquercetin has another name of taxifolin, is also called taxifoliol, taxifolin, dihydroguercetin or (2R, 3R)-dihydroquercetin, is an important flavanonol compound existing in a natural world and belongs to a vitamin PP group. The dihydroquercetin has a molecular formula of C₁₅H₁₂O₇, and has a molecular weight of 304.25 and a molecular formula of

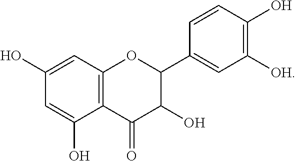 [SMILES: O=C1c2c(O)cc(O)cc2OC(c2ccc(O)c(O)c2)C1O]

As another preferred embodiment of the present disclosure, the flavonoid compound are selected from rutoside, apigenin, apiin, diosmetin or diosmin, wherein

the rutoside is also called rutin, is rutinoside of the flavonoid quercetin, is a flavonoid extracted from plants, and exists in rue, tartary buckwheat, sophora japonica buds, catalpa bungei leaves, tomato stems, leaves, seed shells and the like. The content in dried buckwheat to be bloomed is about 3%, and the rutin is contained in Chinese-dates, hawthorn, gingko, lycium barbarum, motherwort, radix bupleuri, selfheal, aloe, gynostemma pentaphylla and the like. The content of the rutin in buds of sophora japonica of sophorajaponicaL can reach 20% or higher, and the buds of sophora japonica of sophorajaponicaL are main raw materials for extracting the rutin in the medical industry in China. The rutoside has a molecular formula of C₂₇H₃₀O₁₆ and a structural formula of

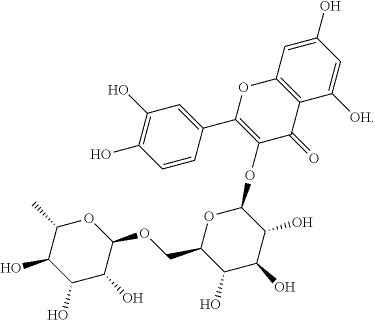 [SMILES: C[C@@H]1O[C@@H](OC[C@H]2O[C@@H](Oc3c(-c4ccc(O)c(O)c4)oc4cc(O)cc(O)c4c3=O)[C@H](O)[C@@H](O)[C@@H]2O)[C@H](O)[C@H](O)[C@H]1O]

The apigenin is a natural existing flavonoid, exists in multiple plants in a form of plant xanthophylls, mainly comes from umbelliferae celery, and also exists in other plants such as chamomile, Melissa, purple perilla, verbena and yarrow. The apigenin has a molecular formula of C₁₅H₁₀O₅ and a structural formula of

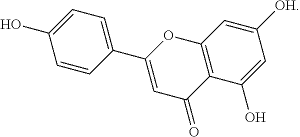 [SMILES: O=c1cc(-c2ccc(O)cc2)oc2cc(O)cc(O)c12]

The apiin is also called apigenin, apiin, apigenin-7-O-glucose-2-O-apigenin, and has a molecular formula of C₂₆H₂₈O₁₄, and a structural formula of

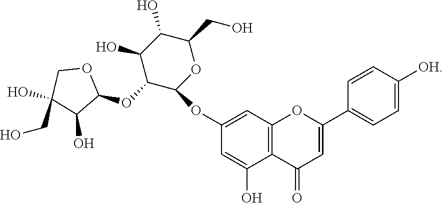 [SMILES: O=c1cc(-c2ccc(O)cc2)oc2cc(O[C@@H]3O[C@H](CO)[C@@H](O)[C@H](O)[C@H]3O[C@@H]3OC[C@](O)(CO)[C@@H]3O)cc(O)c12]

The diosmetin is a monomer extracted from lemon, is contained in many natural plants such as spearmint, valeriana jatamansi and the like, belongs to flavonoids, and has a molecular formula of C₁₆H₁₂O₆ and a structural formula of

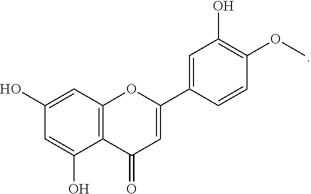 [SMILES: COc1ccc(-c2cc(=O)c3c(O)cc(O)cc3o2)cc1O]

The diosmin is also called diosmin, 3′,5,7-trihydroxy-4′-methoxyflavone, 7-rutionose, buchu resi and diosmin, and has a molecular formula of C₂₈H₃₂O₁₅ and a structural formula of

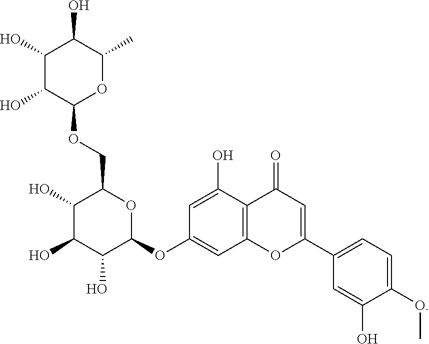 [SMILES: COc1ccc(-c2cc(=O)c3c(O)cc(O[C@@H]4O[C@H](CO[C@@H]5O[C@@H](C)[C@H](O)[C@@H](O)[C@H]5O)[C@@H](O)[C@H](O)[C@H]4O)cc3o2)cc1O]

The flavonoid compound in the present disclosure are only part of preferred types. A flavonoid compounds with the effects of decreasing capillary permeability are not completely illustrated known by those skilled in the art. For a treatment mechanism of the present disclosure, all flavonoid compound with the effects of decreasing the capillary permeability may be applied to the present disclosure.

In the present disclosure, the calcium comes from a composition of more than one of dairy products, bean products, grains, eggshells, shells, seaweeds and bone substances. Preferably, the calcium in the present disclosure is milk calcium, eggshell calcium, oyster calcium, seaweed calcium, coral calcium, pearl calcium, bone calcium, calcium lactate, calcium acetate, calcium gluconate, calcium citrate, calcium phosphate, calcium carbonate, calcium pantothenate or calcium chloride. Most preferably, the calcium is the milk calcium which comes from the dairy products.

The vitamin C in the present disclosure is preferably natural vitamin C, e.g., natural vitamin C extracted from cherries, kiwi fruits, jujubes, oranges, lemons, grapefruits, grapes, apples, persimmons, pomegranate, blackberries, strawberries, apricots, hawthorn and the like, and the safety is easy to be ensured.

The calcium and the vitamin C are common nutritional ingredients in health foods. According to the recommended standard of Chinese Society of Nutrition, calcium intake of an adult per day is 800-1000 mg, and the highest tolerance limit is 2000 mg. Intake of the vitamin C of an adult per day should be 100 mg, and a harmful limit is 1000 mg; the content of the vitamin C in the drug is generally controlled to be 50-500 mg. When taken alone, the calcium or the vitamin C is only limited to treat mild or early androgenetic alopecia, and is not suggested to be continuously taken for a long time. A dose of the vitamin C per day in the present disclosure is 100-300 mg, and the vitamin C should be continuously taken for less than or equal to 12 months. A dose of the calcium per day in the present disclosure is 300-700 mg, and the calcium should be continuously taken for less than or equal to 12 months. A dose of the flavonoid compound per day in the present disclosure is 100-250 mg, and the flavonoid compound should be continuously taken for less than or equal to 6 months.

As a preferred embodiment of the present disclosure, the active ingredients of the drug for preventing and treating alopecia in the present disclosure comprise the flavonoid compound and calcium, wherein weight of the flavonoid compound is less than ⅓ of total weight of the active ingredient; and more preferably, a weight ratio of the flavonoid compound to the calcium is 1:(2-10).

As another preferred embodiment of the present disclosure, the active ingredients of the drug for preventing and treating alopecia comprise the flavonoid compound and the vitamin C, wherein a mass ratio of the flavonoid compound to the total active ingredients is less than ½; and preferably, a weight ratio of the flavonoid compound to the vitamin C is 1:(1-5).

According to combined matching, usage ratios are defined of the flavonoid compound to the vitamin C and the flavonoid compound to the calcium in the present disclosure, thereby ensuring long-term using safety and achieving excellent treatment effects.

As the most preferable embodiment of the present disclosure, the active ingredient of the drug for preventing and treating alopecia in the present disclosure comprise the flavonoid compound, the calcium and the vitamin C, wherein the mass ratio of the flavonoid compound to the total active ingredients is less than ⅓, i.e., when the flavonoid compound, the calcium and the vitamin C are combined to be used, a dosage of the flavonoid compound should not be more than ⅓ of the total amount of the active ingredients. As an optimization of embodiments, a specific solution for proportioning the above active ingredients is given in the present disclosure, i.e., the active ingredient of the drug for preventing and treating alopecia comprise the flavonoid compound, the calcium and the vitamin C, wherein a weight ratio of the flavonoid compound to the calcium and to the vitamin C is 1:(1-10):(1-10); and more preferably, the weight ratio of the flavonoid compound to the calcium and to the vitamin C is 1:(2-4):(2.5-5). As an optimization of embodiments, a more specific solution for proportioning the above active ingredients is given in the present disclosure, i.e., the active ingredients of the drug for preventing and treating alopecia comprise the quercetin, the milk calcium and the vitamin C, wherein a weight ratio of the quercetin to the milk calcium and to the vitamin C is 1:(1-10):(1-10), and preferably, the weight ratio of the quercetin to the milk calcium and to the vitamin C is 1:(2-4):(2.5-5).

In the present disclosure, the needed dose of the composition for treatment is related to many factors including specific applications, properties of the used composition, disease states for treatment, administration manners and conditions of patients. Generally, when the drug for preventing and treating the alopecia is taken in the present disclosure in a form of tablets, each tablet contains 10-40 mg of the flavonoid compound, 30-100 mg of the calcium and 30-100 mg of the vitamin C. 3-7 tablets are taken for a male adult per day, and 2-4 tablets for a female adult per day. When the three substances are combined to be used, the drug is suitable for all patients suffering from the androgenetic alopecia, high in safety and obvious in treatment effect and can be taken for a long time.

In addition, those skilled in the art should know that the flavonoid compound extracted from the plants are often not pure products, and even if the products are separated and purified, the purity often does not reach 100%. The weight ratio described in the present disclosure refers to a weight ratio of a pure product of a certain active ingredient. During preparation, the weight of the extracted or separated and purified flavonoid compound should be converted into weight of the purified active ingredient for calculation.

The present disclosure further provides a preparation for preventing and treating alopecia and promoting hair growth. The preparation may only contain the following active ingredients: a composition including more than one of flavonoid compound with effects of decreasing capillary permeability, calcium and vitamin C, and may also simultaneously contain one or more carriers or excipients.

During preparation of the above preparation for preventing and treating alopecia and promoting hair growth, the following active ingredients: the composition including more than one of the flavonoid compound with the effects of decreasing the capillary permeability, the calcium and the vitamin C, may be mixed with conventional pharmaceutically-acceptable carriers and/or excipients well-known by those skilled in the art.

The carriers must be pharmaceutically acceptable, which is compatible with any other components in the preparation, and must be harmless to a patient. The carriers or excipients may be solids or liquid, or both, and are preferably prepared into a preparation with the compound of a unit dose. For example, a tablet may include an active ingredient with a weight ratio of 0.1-59%, or an active ingredient with a weight ratio of 100%. The preparation in the present disclosure may be prepared by using any well-known pharmaceutical technology which basically includes a step of mixing the components, and the preparation optionally includes one or more auxiliary components.

The preparation in the present disclosure is generally suitable for oral administration. The preparation suitable for oral administration may be prepared into a single unit, such as a capsule, a cachet, a troche or a tablet, wherein each unit contains an active ingredient of a preset dose. The preparation may be further prepared into powder or granules, solution or suspension in water or non-aqueous liquid, oil-in-water emulsion or water-in-oil emulsion. These preparations may be prepared by using an appropriate pharmacologic method, including a step of mixing the active ingredient with an appropriate carrier. Generally, the preparation in the present disclosure is prepared as follows. The active ingredient is uniformly and tightly mixed with liquid or a fine disperse solid carrier or with the liquid and the solid carrier, and the obtained mixture is shaped, e.g., formed into a preparation in a form of a unit dose. For example, the tablets may be prepared by tableting or casting powder or granules containing the active ingredient, wherein the powder or granules may optionally contain one or more auxiliary components. The tableting may be realized by pressing freely-flowing compounds, such as the powder or the granules in an appropriate machine, wherein the powder or the granules may be optionally mixed with an adhesive, a lubricating agent, an inert diluent and/or a surfactant/dispersing agent. Molded tablets may be prepared by casting a powdered compound wetted by an inert liquid adhesive in the appropriate machine.

In addition to an oral administration manner, the present disclosure is applicable to being prepared as dermal topical administration, and preferably prepared into ointments, cream, lotion, paste, gel, spray, aerosol or oil. Usable carriers include vaseline, wool fat, polyethylene glycol, ethanol and a composition of two or more thereof. A concentration of the active ingredients is generally 0.1-5% in weight, e.g., 0.5-2%. Examples of these compositions include cosmetic cream for skin.

The active ingredient of the present disclosure may be further provided in a form of food. For example, the active ingredient is added into the food in ways of adding, mixing, coating, binding or others. The term “food” includes liquid food in the broadest sense, e.g., including drink including dairy products, and other food including healthy bar food, desserts and the like. Food containing the compounds in the present disclosure may be prepared according to a conventional method.

Some preferred embodiments are listed below to illustrate essential contents of the present disclosure.

| | | | | Weight ratio (B converted |
|---|---|---|---|---|
| | | | | into pure calcium for |
| Number/Component | A | B | C | calculation) |
| Embodiment 1 | Quercetin | Milk | — | A:B = 1:4 |
| | | calcium | | |
| Embodiment 2 | Quercetin | — | Vitamin C | A:C = 1:5 |
| Embodiment 3 | Quercetin | Milk | Vitamin C | A:B:C = 1:1:1 |
| | | calcium | | |
| Embodiment 4 | Quercetin | Milk | Vitamin C | A:B:C = 1:2.5:3 |
| | | calcium | | |
| Embodiment 5 | Quercetin | Milk | Vitamin C | A:B:C = 1:3:4 |
| | | calcium | | |
| Embodiment 6 | Quercetin | Calcium | Vitamin C | A:B:C = 1:4:6 |
| | | gluconate | | |
| Embodiment 7 | Quercetin | Calcium | Vitamin C | A:B:C = 1:6:5 |
| | | gluconate | | |
| Embodiment 8 | Quercetin | Calcium | Vitamin C | A:B:C = 1:3:3 |
| | | phosphate | | |
| Embodiment 9 | Kaempferol | Milk | Vitamin C | A:B:C = 1:7:2 |
| | | calcium | | |
| Embodiment 10 | Kaempferol | Milk | — | A:B = 1:3 |
| | | calcium | | |
| Embodiment 11 | Kaempferol | Milk | Vitamin C | A:B:C = 1:2.5:3 |
| | | calcium | | |
| Embodiment 12 | Kaempferol | Calcium | Vitamin C | A:B:C = 1:2:2 |
| | | gluconate | | |
| Embodiment 13 | Kaempferide | Calcium | Vitamin C | A:B:C = 1:1.5:4 |
| | | citrate | | |
| Embodiment 14 | Kaempferide | Calcium | Vitamin C | A:B:C = 1:5:7 |
| | | citrate | | |
| Embodiment 15 | Kaempferide | Calcium | Vitamin C | A:B:C = 1:8:3.5 |
| | | acetate | | |
| Embodiment 16 | Kaempferide | Calcium | Vitamin C | A:B:C = 1:5:2 |
| | | phosphate | | |
| Embodiment 17 | Kaempferide | Calcium | Vitamin C | A:B:C = 1:7.5:8 |
| | | phosphate | | |
| Embodiment 18 | Dihydroquercetin | Calcium | Vitamin C | A:B:C = 1:2:5.5 |
| | | carbonate | | |
| Embodiment 19 | Dihydroquercetin | Milk | Vitamin C | A:B:C = 1:9:10 |
| | | calcium | | |
| Embodiment 20 | Dihydroquercetin | — | Vitamin C | A:C = 1:4 |
| Embodiment 21 | Dihydroquercetin | Milk | Vitamin C | A:B:C = 1:2.5:3 |
| | | calcium | | |
| Embodiment 22 | Rutoside | Milk | Vitamin C | A:B:C = 1:2.5:3 |
| | | calcium | | |
| Embodiment 23 | Apigenin | Milk | Vitamin C | A:B:C = 1:2.5:3 |
| | | calcium | | |
| Embodiment 24 | Apiin | Milk | Vitamin C | A:B:C = 1:2.5:3 |
| | | calcium | | |
| Embodiment 25 | Diosmetin | Milk | Vitamin C | A:B:C = 1:2.5:3 |
| | | calcium | | |
| Embodiment 26 | Diosmin | Milk | Vitamin C | A:B:C = 1:2.5:3 |
| | | calcium | | |

The drug for preventing and treating alopecia listed in the above table in the present disclosure is suitable for a patient suffering from the androgenetic alopecia. When the drug is taken orally, a hair loss phenomenon may be obviously controlled within 1-2 weeks. Within 1-4 months after oral administration, new hair may grow on the head of the patient, hair quality and color of the new hair are natural. Length of the hair is about 10 mm, and density of the hair is close to or equivalent to density of original hair. Within 6-12 months after oral administration, obviously lengthened new hair may be seen, the length of the hair reaches 5 cm or more, the hair covers a hair loss part totally. Hair quality of the new hair is natural, and a color of the new hair is close to or the same as that of the original hair.

The applicant counts treatment data from January, 2003 to June, 2016. All patients accepting treatment are patients suffering from the androgenetic alopecia. A number of the patients is 50, including 46 male patients and 4 female patients. An age range of the patients is between 24 and 60, and courses of disease are from 1 to 10 years. The patients orally take the tablets. Tracking and return visit results show that administration courses of 50 patients are unequal from 3 months to 10 years. When the composition in the present disclosure is taken by 100% of the patients, the original hair loss is completely controlled, and growth of the new hair is good, so that the top of the head with originally sparse hair, half-baldness or total baldness restores vitality, and any obvious adverse reaction is not present.

The drug for preventing and treating the alopecia provided by the present disclosure can achieve an aim of completely fundamentally curing the androgenetic alopecia, and is safe and effective when persistently taken for a long time. Some typical cases are listed below to describe curative effects of the present disclosure.

Case 1

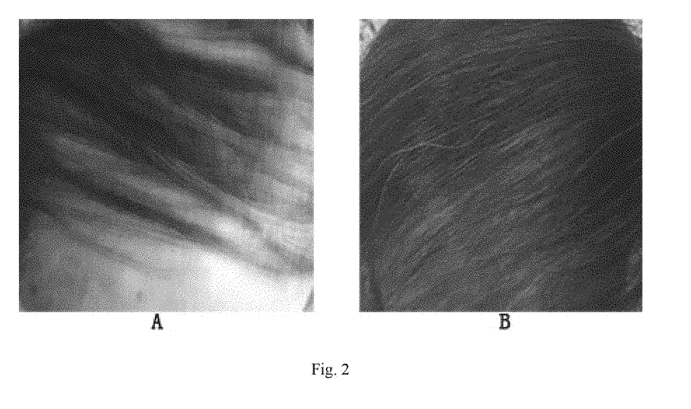

Mr Hua, male, 52 years old, from Wolong District, Nanyang City, who has been suffered from the androgenetic alopecia since 2005 and have been treated with traditional Chinese medicine for many years. The androgenetic alopecia is not effectively controlled by continuously using multiple methods. Hair is still continuously lost, and new hair does not obviously grow. In March, 2013, hair on the forehead of the patient is basically completely lost, and hair on the top of the head is sparse. Through oral medication of the composition in embodiment 4 of the present disclosure, obvious new hair grows on the forehead and the top of the head before the end of 2014. Hair quality is natural, and a color of the hair is normal. In March 2015, the new hair is obviously lengthened, is thicker and basically covers the scalp. The hair is maintained at a certain base number and is not lost any more since the drug is taken up to now, and any obvious adverse reaction is not present in a period of taking the drug. FIG. 2(A) is a photo taken by the patient in March, 2013, and FIG. 2(B) is a photo taken by the patient in March, 2015. Obvious treatment effects can be seen by comparing the A and the B in FIG. 2.

Case 2

Mrs Shen, female, 43 years old, from Wancheng District, Nanyang City, who has been suffered from abnormal alopecia in 2008. Her hair loss situation is sometimes good and sometimes bad. Hair on the top of the head is obviously decreased in 2010 and is fine and soft. The patient has started to seek treatment since March, 2010 and has been treated by Western medicine and traditional Chinese medicine intermittently for 3 years. In August, 2013, the patient was treated by the formular of the present disclosure. At this moment, hair loss conditions of the patient are severer compared with those in 2010, and no sign of improvement is present. When subjected to oral medication by adopting the composition in embodiment 5 of the present disclosure, the hair loss is controlled in March, 2014, and new hair grows out. At the end of 2014, hair on the top of the head is obviously increased and is thicker, hair quality is close to that of the original hair, and any adverse reaction is not caused in the drug administration period.

Case 3

Mr Zhang, male, 46 years old, from Wancheng District, Nanyang City, who has been suffered from alopecia from an age of 31. Hair is lost from temples on two sides of the forehead, and then lost upwards in an M shape. Hair on the top of the head is basically completely lost at an age of 36. Multiple treatment methods are adopted in a hair loss period, but loss of a great amount of hair cannot be effectively prevented. In July, 2009, the patient is treated by the formular of the present disclosure. At this moment, hair on the top of the head of the patient is completely lost, and the treatment is abandoned for more than 1 year. By adopting oral medication of the composition in embodiment 4 of the present disclosure, obviously thick hair grows out on the top of the head in June, 2010, and the hair quality is natural. Newly growing hair basically covers a hair loss region at the end of 2010, and any adverse reaction is not caused in the drug administration period.

The above cases are only part of medical treatment cases for the present disclosure, which are true cases and are only used for describing part of specific indications in the present disclosure. However, the present disclosure is not limited to symptoms reflected in the above cases. Application of the present disclosure in treatment of other diseases by combining with conventional knowledge by those skilled in the art according to contents of the present disclosure is also included in a protection scope of the present disclosure.

Finally, it should be noted that, the above embodiments are only used for describing, not for limiting the technical solutions of the present disclosure. Other modifications or equivalent replacements made to the technical solutions of the present disclosure by those ordinary skilled in the art should be included in the scope of claims of the present disclosure on premise of not deviating from the spirit and scope of the technical solutions of the present disclosure.

What is claimed is:

1. A drug for preventing and treating alopecia, comprising following active ingredients:
a composition including more than one of a flavonoid compound with effects of decreasing capillary permeability, calcium and vitamin C.

2. The drug for preventing and treating alopecia according to claim 1, wherein
the flavonoid compound is quercetin, kaempferol or kaempferide.

3. The drug for preventing and treating alopecia according to claim 1, wherein
the flavonoid compound is dihydroquercetin.

4. The drug for preventing and treating alopecia according to claim 1, wherein
the flavonoid compound is rutin, apigenin, apiin, diosmetin or diosmin.

5. The drug for preventing and treating alopecia according to claim 1, wherein
the calcium is milk calcium, eggshell calcium, oyster calcium, seaweed calcium, coral calcium, pearl calcium, bone calcium, calcium lactate, calcium acetate, calcium gluconate, calcium citrate, calcium phosphate, calcium carbonate, calcium pantothenate or calcium chloride.

6. The drug for preventing and treating alopecia according to claim 1, wherein
the active ingredients of the drug for preventing and treating alopecia consist of the flavonoid compound and the calcium, wherein
a mass ratio of the flavonoid compound to total active ingredients is less than ⅓.

7. The drug for preventing and treating alopecia according to claim 1, wherein
the active ingredients of the drug for preventing and treating alopecia consist of the flavonoid compound and the vitamin C, wherein
a mass ratio of the flavonoid compound to the total active ingredients is less than ½.

8. The drug for preventing and treating alopecia according to claim 1, wherein
the active ingredients of the drug for preventing and treating alopecia consist of the flavonoid compound, the calcium and the vitamin C, wherein
a mass ratio of the flavonoid compound to the total active ingredients is less than ⅓.

9. The drug for preventing and treating alopecia according to claim 8, wherein
the active ingredients of the drug for preventing and treating alopecia comprise the flavonoid compound, the calcium and the vitamin C, wherein
a mass ratio of the flavonoid compound to the calcium and to the vitamin C is 1:(1-10):(1-10).

10. The drug for preventing and treating alopecia according to claim 9, wherein
the active ingredients of the drug for preventing and treating alopecia consist of the quercetin, the milk calcium and the vitamin C, wherein
a mass ratio of the quercetin to the milk calcium and the vitamin C is 1:(1-10):(1-10).

11. The drug for preventing and treating alopecia according to claim 2, wherein
the active ingredients of the drug for preventing and treating alopecia consist of the flavonoid compound and the calcium, wherein
a mass ratio of the flavonoid compound to the total active ingredients is less than ⅓.

12. The drug for preventing and treating alopecia according to claim 3, wherein
the active ingredients of the drug for preventing and treating alopecia consist of the flavonoid compound and the calcium, wherein
a mass ratio of the flavonoid compound to the total active ingredients is less than ⅓.

13. The drug for preventing and treating alopecia according to claim 4, wherein
the active ingredients of the drug for preventing and treating alopecia consist of the flavonoid compound and the calcium, wherein
a mass ratio of the flavonoid compound to the total active ingredients is less than ⅓.

14. The drug for preventing and treating alopecia according to claim 5, wherein
the active ingredients of the drug for preventing and treating alopecia consist of the flavonoid compound and the calcium, wherein
a mass ratio of the flavonoid compound to the total active ingredients is less than ⅓.

15. The drug for preventing and treating alopecia according to claim 2, wherein
the active ingredients of the drug for preventing and treating alopecia consist of the flavonoid compound and the vitamin C, wherein
a mass ratio of the flavonoid compound to the vitamin C is less than ½.

16. The drug for preventing and treating alopecia according to claim 3, wherein
the active ingredients of the drug for preventing and treating alopecia consist of the flavonoid compound and the vitamin C, wherein
a mass ratio of the flavonoid compound to the total active ingredients is less than ½.

17. The drug for preventing and treating alopecia according to claim 4, wherein
the active ingredient of the drug for preventing and treating alopecia consist of the flavonoid compound and the vitamin C, wherein
a mass ratio of the flavonoid compound to the total active ingredients is less than ½.

18. The drug for preventing and treating alopecia according to claim 5, wherein
the active ingredients of the drug for preventing and treating alopecia consist of the flavonoid compound and the vitamin C, wherein
a mass ratio of the flavonoid compound to the total active ingredients is less than ½.

19. The drug for preventing and treating alopecia according to claim 2, wherein
the active ingredients of the drug for preventing and treating alopecia consist of the flavonoid compound, the calcium and the vitamin C, wherein
a mass ratio of the flavonoid compound to the total active ingredients is less than ⅓.

20. The drug for preventing and treating alopecia according to claim 3, wherein
the active ingredient of the drug for preventing and treating alopecia consist of the flavonoid compound, the calcium and the vitamin C, wherein
a mass ratio of the flavonoid compound to the total active ingredients is less than ⅓.
